(19) United States
(12) Patent Application Publication
(10) Pub. No.: US 2014/0100437 A1
(43) Pub. Date: Apr. 10, 2014

(54) NON-INVASIVE DIAGNOSTIC METHOD FOR BREAST CANCER

(71) Applicants: Chang Gung Medical Foundation Chang Gung Memorial Hospital, Linkou, Taoyuan (TW); Industrial Technology Research Institute, Hsinchu (TW)

(72) Inventors: Shih-Bin Luo, Hsinchu County (TW); De-Yi Chiou, New Taipei City (TW); Wan-Ting Tien, Chiayi City (TW); Meng-Lin Li, Hsinchu City (TW); Shin-Cheh Chen, Hsinchu City (TW)

(21) Appl. No.: 13/943,801

(22) Filed: Jul. 17, 2013

Related U.S. Application Data
(60) Provisional application No. 61/709,598, filed on Oct. 4, 2012.

(51) Int. Cl. A61B 5/00; A61B 6/00

(52) U.S. Cl. CPC A61B 5/0095; A61B 6/502
USPC 600/407

(57) ABSTRACT

This disclosure provides a photoacoustic imaging method for calcifications or microcalcifications. This photoacoustic imaging method is able to determine benign or malignant calcifications in a non-invasive way.

### CROSS-REFERENCE TO RELATED APPLICATION

This application claims the priority benefits of U.S. provisional application Ser. No. 61/709,598, filed on Oct. 4, 2012. The entirety of the above-mentioned patent application is hereby incorporated by reference herein and made a part of this specification.

### TECHNICAL FIELD

The present disclosure relates to a diagnostic method. More particularly, the present disclosure relates to a non-invasive diagnostic method for breast cancer.

### BACKGROUND

Breast cancer is one of the most common cancers occurring in women and almost accounts for a quarter of the cancers in women. According to the statistics issued by the authorities, around tenth of the breast cancer patients is diagnosed with ductal carcinoma in situ (DCIS, also called stage zero breast cancer). As the occurrence of ductal carcinoma in situ (DCIS) is increasing in recent years, it is important to diagnose DCIS at early stage by identifying the breast calcifications.

High-quality x-ray mammography is a valuable diagnostic tool for identifying the breast calcifications, as the breast microcalcifications appear as fine white specks or flecks on the mammograms. However, mammography is limited in evaluating calcifications to be benign or malignant microcalcifications. Also, the inconveniences, discomforts and radiation of the mammography put a limit to this technology.

Breast ultrasound, also known as sonography, is also a useful tool for breast cancer screening. Usually, breast ultrasound is used to target a specific area of concern found on the mammogram and ultrasound may help distinguish between cysts and solid masses. However, many calcifications seen on mammography cannot be seen on ultrasound, so that certain early breast cancers only shown as calcifications on mammography may be overlooked.

In general, if the pattern of the microcalcifications are suspicious, further invasive tests, such as the needle localization biopsy, or additional expensive imaging tests may be necessary, in order to determine the calcifications to be benign or malignant.

### SUMMARY

As embodied and broadly described herein, a photoacoustic imaging method for identifying calcification or microcalcification in target tissues is provided. The photoacoustic imaging method comprises irradiating a laser pulse of a first wavelength to the target having a first type of calcification and/or a second type of calcification to induce a first photoacoustic signal from the first type of calcification and/or a second photoacoustic signal from the second type of calcification. The first photoacoustic signal and/or the second photoacoustic signal may be received to form a photoacoustic image. The first photoacoustic signal forms a first calcification pattern in the photoacoustic image showing locations of the first type of calcification, while the second photoacoustic signal forms a second calcification pattern in the photoacoustic image showing locations of the second type of calcification. The photoacoustic image is then analyzed to verify the locations of the first type of calcification and the second type of calcification.

As embodied and broadly described herein, a photoacoustic imaging method for identifying calcification or microcalcification in target tissues is provided. The photoacoustic imaging method comprises irradiating a laser pulse of a first wavelength to the target having a first type of calcification and/or a second type of calcification to induce a first photoacoustic signal from the first type of calcification and/or a second photoacoustic signal from the second type of calcification. The photoacoustic imaging method comprises irradiating a laser pulse of a second wavelength to the target having a first type of calcification and/or a second type of calcification to induce a third photoacoustic signal from the first type of calcification and/or a fourth photoacoustic signal from the second type of calcification The first photoacoustic signal and/or the second photoacoustic signal may be received to form a first photoacoustic image, wherein the first photoacoustic signal forms a first calcification pattern in the first photoacoustic image showing locations of the first type of calcification, while the second photoacoustic signal forms a second calcification pattern in the first photoacoustic image showing locations of the second type of calcification. The third photoacoustic signal and/or the fourth photoacoustic signal may be received to form a second photoacoustic image, wherein the third photoacoustic signal forms a third calcification pattern in the second photoacoustic image showing locations of the first type of calcification, while the fourth photoacoustic signal forms a fourth calcification pattern in the second photoacoustic image showing locations of the second type of calcification. The first and second photoacoustic images are analyzed to verify the locations of the first type of calcification and the second type of calcification.

Several exemplary embodiments accompanied with figures are described in detail below to further describe the disclosure in details.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1 is a photoacoustic spectrum of calcium oxalate and calcium phosphate samples.

FIG. 2 is a photoacoustic spectrum of calcium oxalate and calcium phosphate.

FIG. 3 displays the general principle of the photoacoustic imaging method and the obtained photoacoustic image according to one embodiment of this disclosure.

FIG. 4A is a flow chart illustrating the process steps of the photoacoustic imaging method according to one embodiment of this disclosure.

FIG. 4B is a flow chart illustrating the diagnosis of breast calcification using the photoacoustic imaging method of this disclosure.

FIGS. 5A-5B display the general principle of the photoacoustic imaging method and the obtained photoacoustic images according to another embodiment of this disclosure.

FIG. 6A is a flow chart illustrating the process steps of the photoacoustic imaging method according to another embodiment of this disclosure.

FIG. 6B is a flow chart illustrating the diagnosis of breast calcification using the photoacoustic imaging method of this disclosure.

FIGS. 7A-7C display the general principles of the photoacoustic imaging method and the obtained photoacoustic images according to another embodiment of this disclosure.

FIG. 7D is a flow chart illustrating the diagnosis of breast calcification using the photoacoustic imaging method of this disclosure.

FIG. 8 is a photoacoustic spectrum of calcium phosphate samples and blood vessel.

FIG. 9 is a photoacoustic spectrum of calcium phosphate and calcium oxalate.

FIG. 10 is a flow chart illustrating the process steps of the photoacoustic imaging method according to another embodiment of this disclosure.

### DETAILED DESCRIPTION OF DISCLOSED EMBODIMENTS

Two types of calcification have been reported in breast tissues. One type of calcification is opaque deposit and has been found to be composed mostly of calcium phosphate (CaP). The other type of calcification is colorless deposit with the presence of calcium oxalate (CaOx). Calcium phosphate is the predominant form of calcium deposits seen in breast tissue and is frequently associated with malignancy. Calcium oxalate, on the other hand, has been reported to be associated with benign lesions. Thus, if the compositions or ingredient of calcifications can be analyzed in a non-invasive way to distinguish calcium phosphate from calcium oxalate, the malignancy or benignancy of the calcification can be determined and biopsy may be avoided for some patients.

Calcium phosphate and calcium oxalate have different translucency. Calcium phosphate is opaque, while calcium oxalate is almost translucent and has a high diopter. The density of calcium phosphate is 2.32 g/cm³, while the density of calcium oxalate is 1.99 g/cm³. The hardness of calcium oxalate is about twice of that of calcium phosphate. Additionally, calcium phosphate and calcium oxalate have different light adsorption efficiency toward different light wavelengths. Based on the Fourier transformed infrared (FT-IR) spectrum of calcium oxalate (e.g. CaC₂O₄) in the previous studies, there are five absorption bands at the frequency of 1646 cm⁻¹ (wavelength 6075.33 nm), 1384 cm⁻¹ (wavelength 7225.43 nm), 1318 cm⁻¹ (wavelength 7587.25 nm), 782 cm⁻¹ (wavelength 12787.72 nm) and 518 cm⁻¹ (wavelength 19305.02 nm). For the infrared spectrum of calcium phosphate (e.g. Ca₃O₈P₂), there are five absorption bands at the frequency of 1456 cm⁻¹ (wavelength 6868.13 nm), 1384 cm⁻¹ (wavelength 7225.43 nm), 1033 cm⁻¹ (wavelength 9680.54 nm), 603 cm−1 (wavelength 16583.75 nm) and 564 cm−1 (wavelength 17730.5 nm). From the measured

Fourier transformed near-infrared (FT-NIR) spectrum of the powders of calcium phosphate and calcium oxalate, different absorption bands are observed for calcium phosphate and calcium oxalate.

Photoacoustic detection technology is developed based on the photoacoustic effect. Laser pulses are irradiated to the aimed target (i.e. biological tissues or even organs) and the energy absorbed by the target is then converted into heat, leading to transient thermoelastic expansion and thus wideband (e.g. MHz) ultrasonic emission. The generated ultrasonic waves are then detected by ultrasonic transducers. The magnitude of the ultrasonic emission (i.e. the photoacoustic signal), which is proportional to the local energy deposition, reveals physiologically specific optical absorption contrast.

P=Γ·μa·H

wherein P represents the magnitude of the photoacoustic signal, Γ (Grüneisen parameter) represents a dimensionless factor of the thermoacoustic conversion efficiency, μa represents the absorption coefficient and H represents the optical energy. For different ingredients having distinctive hardness and/or density, the thermoacoustic conversion efficiency and the light absorption efficiency are not the same, and the magnitude of the photoacoustic signal is dissimilar. Hence, in this disclosure, the difference between the magnitude of the photoacoustic signal (photoacoustic signal magnitude) of calcium oxalate and that of calcium phosphate is utilized, in order to determine the benignancy or malignancy of the microcalcification.

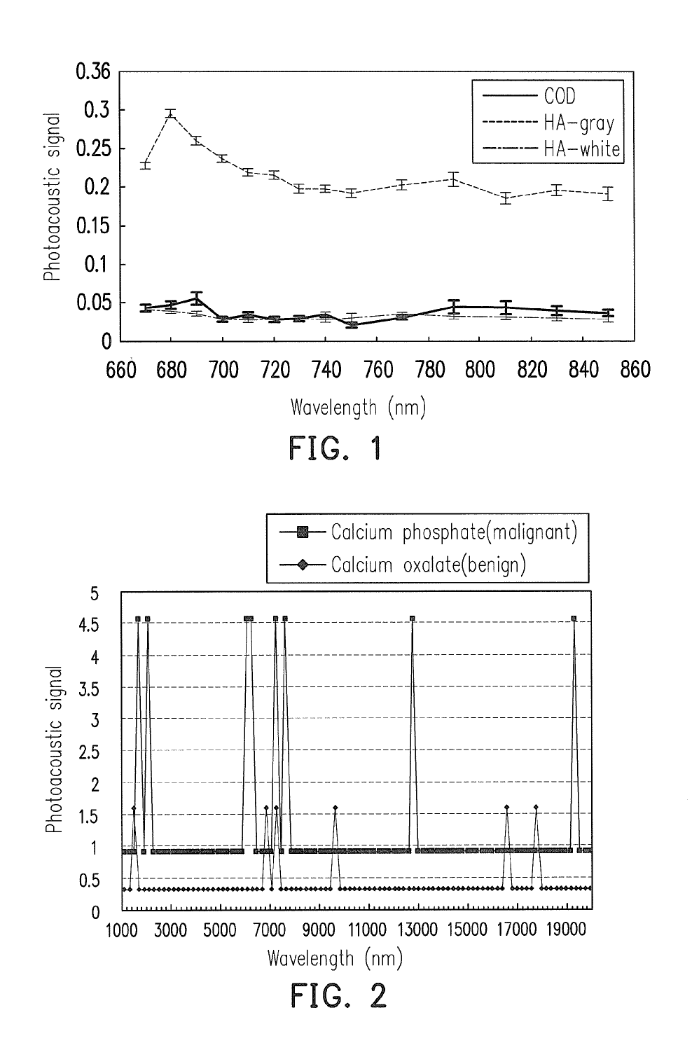

FIG. 1 is a photoacoustic spectrum of calcium oxalate and calcium phosphate samples. The photoacoustic signal magnitudes are recorded as a function of the laser wavelength (the wavelength of the irradiated laser pulse). In FIG. 1, COD represents calcium oxalate sample, HA-gray represents the sintered calcium phosphate sample, and HA-white represents the non-sintered calcium phosphate sample. As shown in FIG. 1, the sintered calcium phosphate sample having a higher hardness exhibits stronger photoacoustic signals, when compared with the non-sintered calcium phosphate sample having a lower hardness. On the other hand, the calcium oxalate sample having a lower density than calcium phosphate exhibits weaker photoacoustic signals. That is, over the laser wavelength of visible/near-infrared light, the magnitudes of the photoacoustic signals for calcium oxalate and calcium phosphate at various wavelengths are different. In FIG. 1, the magnitudes of the photoacoustic signals for calcium phosphate are larger, when compared with calcium oxalate. For the sintered calcium phosphate sample, the strongest photoacoustic signal is observed at 680 nm. However, depending on the laser source available, a laser source capable of generating a laser pulse of 700 nm may be used, for example.

FIG. 2 is a photoacoustic spectrum of calcium oxalate and calcium phosphate. The photoacoustic signal magnitudes are recorded as a function of the laser wavelength over the range of near-infrared/infrared light. By selecting the suitable wavelength of the laser source, it is possible to observe the photoacoustic signal for only one of calcium oxalate and calcium phosphate, as the photoacoustic signal of the other one is rather weak. That is, using the laser of a specific wavelength at which sufficient absorption contrast exists between calcium oxalate and calcium phosphate, the photoacoustic signal between calcium oxalate and calcium phosphate can be differentiated. In general, the photoacoustic spectra of calcium oxalate samples and calcium phosphate samples over the laser wavelengths of visible or near-infrared/infrared light may be obtained in advance, collected as a photoacoustic spectrum databank and used as a reference for selecting the laser wavelength. For example, the possibly used wavelength range of visible light may be 400 nm˜700 nm, and the possibly used wavelength range of near-infrared/infrared may be 650 nm˜950 nm.

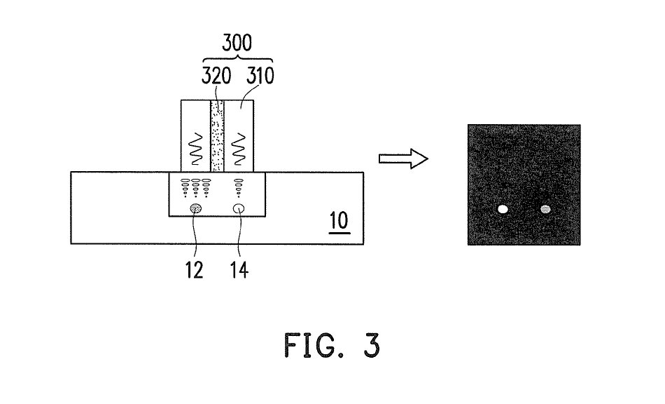

FIG. 3 shows the general principle of the photoacoustic imaging method and the obtained photoacoustic image according to one embodiment of this disclosure. As shown in FIG. 3, the photoacoustic probe 300 comprising at least a laser probe 310 and an ultrasonic sensor or transducer 320 is applied to the target 10. The target 10 may be soft biological tissues or organs with calcification spots or patterns, such as breast, lung or kidney tissues, arteries and thyroid gland. The target 10 comprises a first type of calcification 12 consisting mainly of calcium phosphate and/or a second type of calcification 14 consisting mainly of calcium oxalate. As discussed previously, the first type of calcification 12 may be an indication of malignancy, while the second type of calcification 14 may be an indication of benignancy. The laser (shown as wavy lines) of a specific wavelength is introduced into the target 10 and the photoacoustic signal(s) (ultrasonic emission, shown as segmented triangle) is then detected by the ultrasonic sensor 320 to form the image.

The photoacoustic imaging systems, such as photoacoustic tomography (PAT) and photoacoustic microscopy (PAM), may be used in this disclosure. Taking the photoacoustic microscopy (PAM) as an example, a laser pulse at the wavelength of 700 nm is irradiated to the target tissue to induce acoustic pressure waves, and the photoacoustic signal (ultrasonic emission) is detected by a 50 MHz ultrasound transducer. In the obtained photoacoustic image shown in the right part of FIG. 3, the brighter spot shown in the left side corresponds to calcium phosphate calcification, while the darker spot shown in the right side corresponds to calcium oxalate calcification. This is because calcium phosphate has much stronger optical absorption toward the laser wavelength located in the range of 650 nm to 750 nm, when compared with calcium oxalate. As long as there is sufficient absorption contrast toward the applied laser wavelength, it is possible to differentiate the obtained image of calcium phosphate from the obtained image of calcium oxalate.

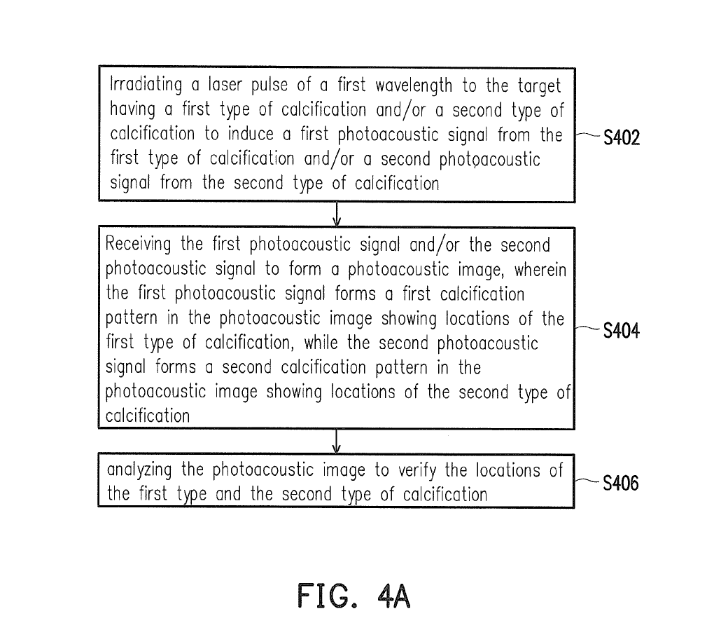

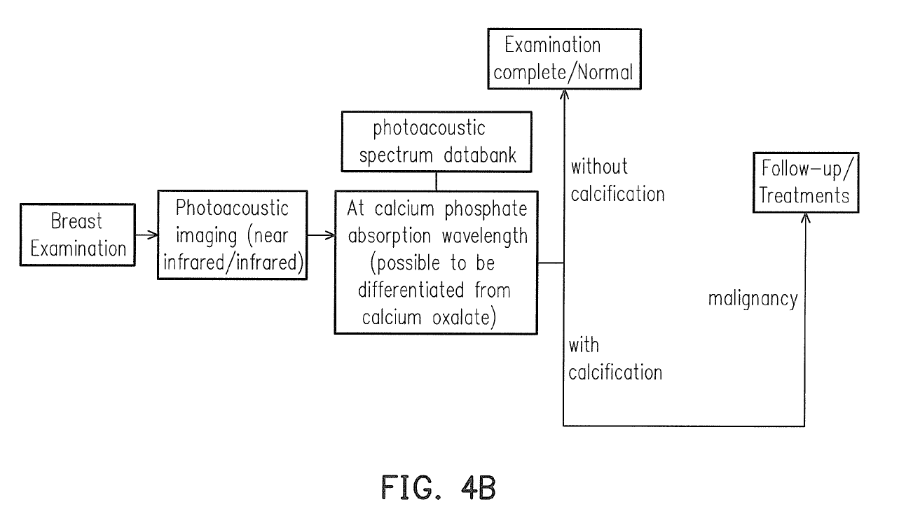

FIG. 4A is a flow chart illustrating the process steps of the photoacoustic imaging method according to one embodiment of this disclosure. In Step S402, a laser pulse of a first wavelength is irradiated to the target having the first type of calcification and/or the second type of calcification to induce a first photoacoustic signal from the first type of calcification and/or a second photoacoustic signal from the second type of calcification. The target may be breast tissues, the first type of calcification is calcium phosphate calcification (i.e. calcification mainly composed of calcium phosphate), and the second type of calcification is calcium oxalate calcification (i.e. calcification mainly composed of calcium oxalate), for example. The first wavelength used in Step S402 is predetermined or elected in advance from the absorption band of the absorption spectrum of calcium phosphate, so that calcium phosphate has strong optical absorption at the first wavelength and calcium oxalate has weak optical absorption at the first wavelength. As the photoacoustic signal is proportional to the optical absorption, the resultant first photoacoustic signal is much stronger than the second photoacoustic signal, if both types of calcification co-exist. In Step S404, the first photoacoustic signal and/or the second photoacoustic signal are received to form a photoacoustic image. The first photoacoustic signal forms a first calcification pattern in the photoacoustic image, showing locations of the first type of calcification. Also, the second photoacoustic signal forms a second calcification pattern in the photoacoustic image, showing locations of the second type of calcification. Later, in Step S406, the photoacoustic image is analyzed to verify the locations of the first type of calcification and the second type of calcification. FIG. 4B is a flow chart illustrating the diagnosis of breast cancer using the aforementioned photoacoustic imaging method of this disclosure. By performing the photoacoustic image method of this disclosure, if no calcification is observed at calcium phosphate absorption wavelength, that is, no malignant calcification is observed, the examination is finished. However, if calcification is observed at calcium phosphate absorption wavelength, that is, malignant calcification is observed, the patient may opt to follow-ups or further treatments.

In the routine breast cancer examination, X-ray mammogram or breast ultrasound (sonogram) is taken and both types of calcification (i.e. calcium phosphate calcification and calcium oxalate calcification) are present in the mammogram or sonogram (ultrasound image). In this case, as only the malignant calcification is shown in the photoacoustic image of this disclosure, the photoacoustic image obtained by using the photoacoustic imaging method of this disclosure may be further compared with the mammogram or ultrasound image for confirmation. Once the malignant calcification is confirmed, the patient may be transferred to further treatments. For example, the ultrasound image (e.g. image obtained from Doppler mode ultrasound) may be used to identify the locations of blood vessels (i.e. the noise or the background signals), which is useful in eliminating the background noise from the photoacoustic images.

However, as both types of calcifications can be differentiated solely by the photoacoustic imaging method of this disclosure, it is not a pre-requisite to acquire sonogram or ultrasound images for comparison with the photoacoustic image of this disclosure in order to determine the benignancy or malignancy of the calcifications.

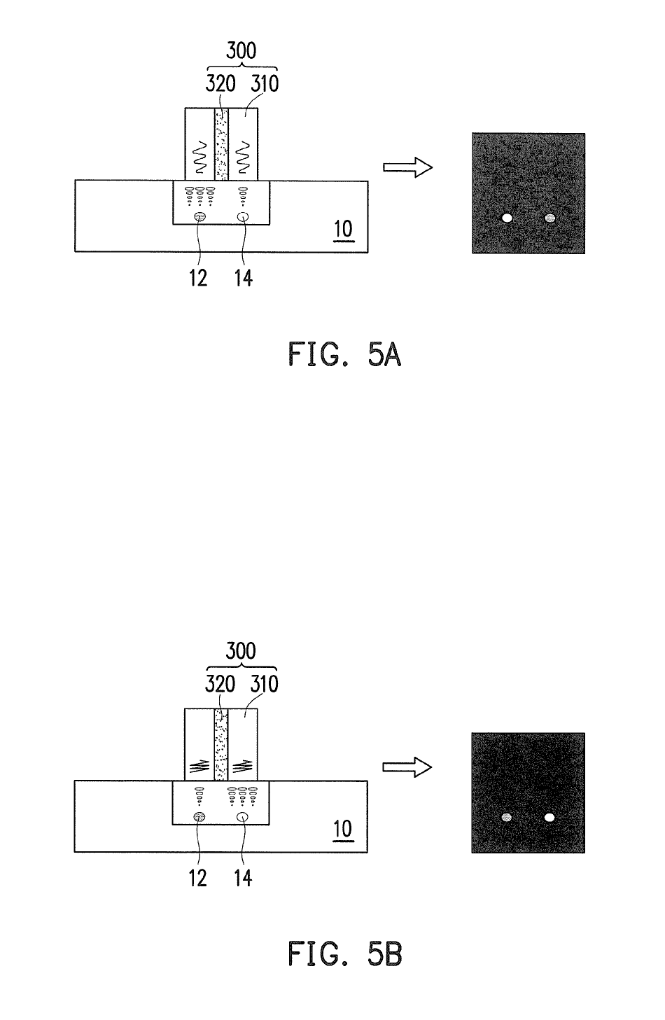

FIGS. 5A-5B show the general principle of the photoacoustic imaging method and the obtained photoacoustic images according to another embodiment of this disclosure. In FIG. 5A, the laser (shown as wavy lines) of a first wavelength is introduced into the target 10 and the obtained photoacoustic image is shown in the right part of FIG. 5A. The brighter spot shown in the left side of FIG. 5A corresponds to calcium phosphate calcification, while the darker spot shown in the right side corresponds to calcium oxalate calcification. This is because calcium phosphate has stronger optical absorption toward the first wavelength, when compared with calcium oxalate. For example, the first wavelength is 700 nm. Also, in FIG. 5B, the laser (shown as wavy lines) of a second wavelength is introduced into the target 10 and the obtained photoacoustic image is shown in the right part of FIG. 5B. The brighter spot shown in the right side of FIG. 5B corresponds to calcium oxalate calcification, while the darker spot shown in the left side corresponds to calcium phosphate calcification. This is because calcium phosphate has weaker optical absorption toward the second wavelength, when compared with calcium oxalate. For example, the second wavelength is 900 nm.

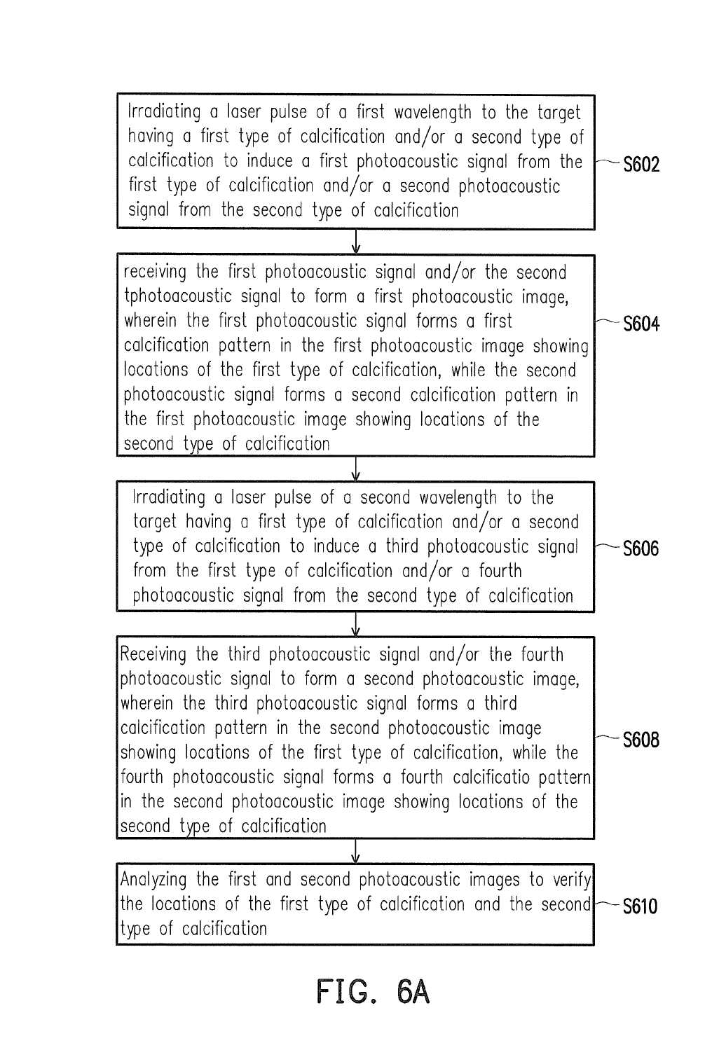

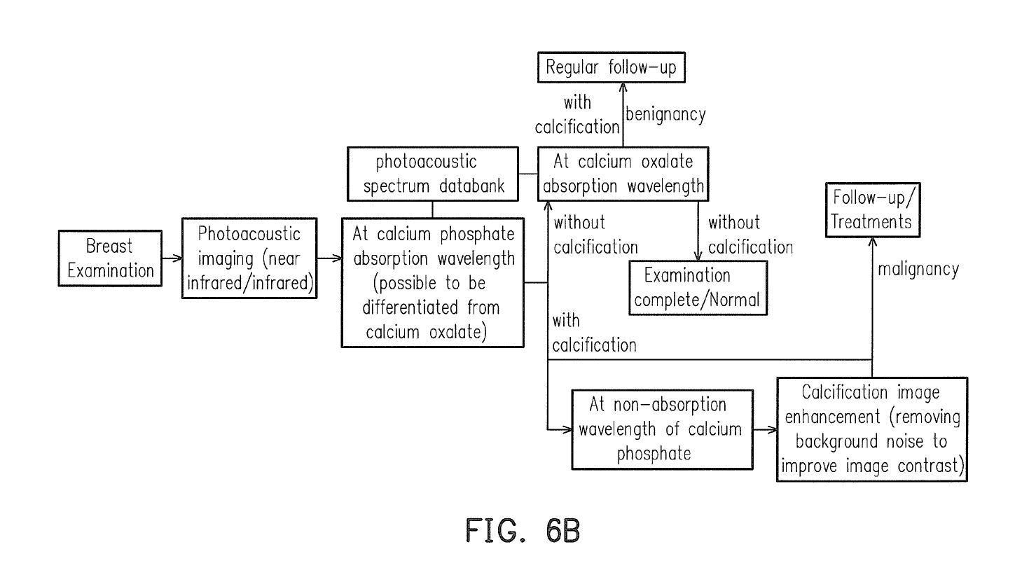

FIG. 6A is a flow chart illustrating the process steps of the photoacoustic imaging method according to another embodiment of this disclosure. In Step S602, a laser pulse of a first wavelength is irradiated to the target having the first type of calcification and/or the second type of calcification to induce a first photoacoustic signal from the first type of calcification and/or a second photoacoustic signal from the second type of calcification. The first wavelength used in Step S602 is predetermined or elected in advance from the absorption band of the absorption spectrum of calcium phosphate, so that calcium phosphate has strong optical absorption at the first wavelength than calcium oxalate. In Step S604, the first photoacoustic signal and/or the second photoacoustic signal are received to form a first photoacoustic image. The first photoacoustic signal forms a first calcification pattern in the first photoacoustic image showing locations of the first type of calcification. Also, the second photoacoustic signal forms a second calcification pattern in the first photoacoustic image showing locations of the second type of calcification.

In Step S606, a laser pulse of a second wavelength is irradiated to the target having the first type of calcification and/or the second type of calcification to induce a third photoacoustic signal from the first type of calcification and/or a fourth photoacoustic signal from the second type of calcification. The second wavelength used in Step S606 is elected in advance from the absorption band of the absorption spectrum of calcium oxalate, so that calcium oxalate has strong optical absorption at the second wavelength than calcium phosphate. In Step S608, the third photoacoustic signal and/or the fourth photoacoustic signal are received to form a second photoacoustic image. The third photoacoustic signal forms a third calcification pattern in the second photoacoustic image, showing locations of the first type of calcification. Also, the fourth photoacoustic signal forms a fourth calcification pattern in the second photoacoustic image, showing locations of the second type of calcification. Later, in Step S610, the first and second photoacoustic images are analyzed to verify the locations of the first type of calcification and the second type of calcification. FIG. 6B is a flow chart illustrating the diagnosis of breast calcification using the aforementioned photoacoustic imaging method of this disclosure. By performing the photoacoustic image method of this disclosure, if no malignant calcification is observed at calcium phosphate absorption wavelength and no calcification is observed at calcium oxalate absorption wavelength, the examination result is normal and the examination is finished. If calcification is observed at calcium oxalate absorption wavelength, that is, benign calcification is observed, the patient may opt to follow-ups. However, if malignant calcification is observed at calcium phosphate absorption wavelength, the patient may opt to follow-ups or further treatments or the aforementioned photoacoustic imaging method may be performed at other non-absorption wavelength of calcium phosphate to enhance the calcification signal by eliminating the noise or background signals, which further confirms the observation of malignant calcification.

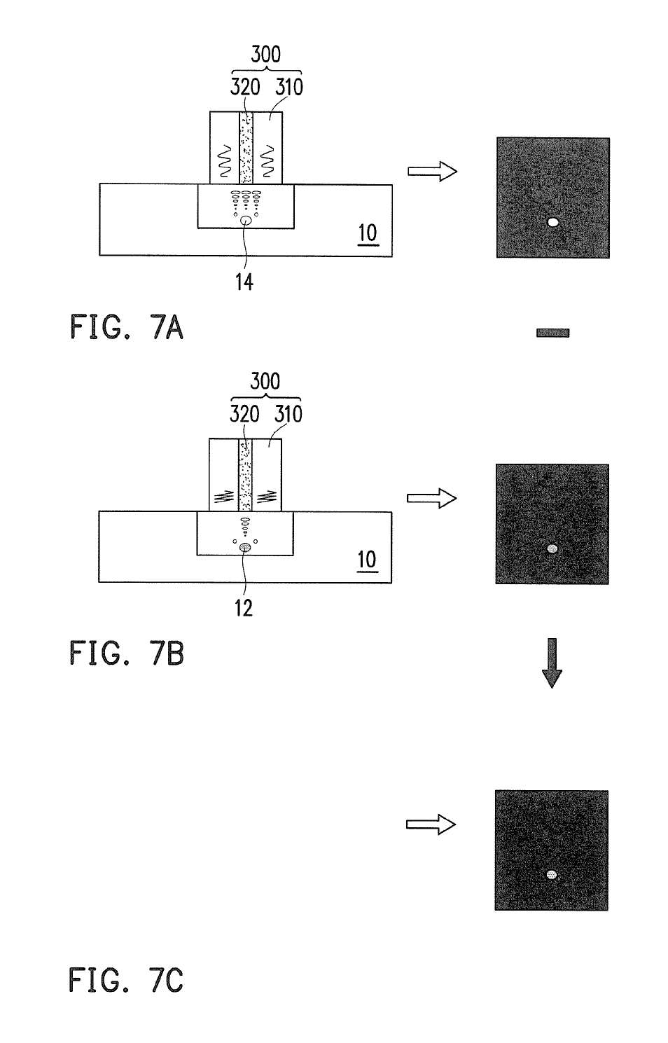

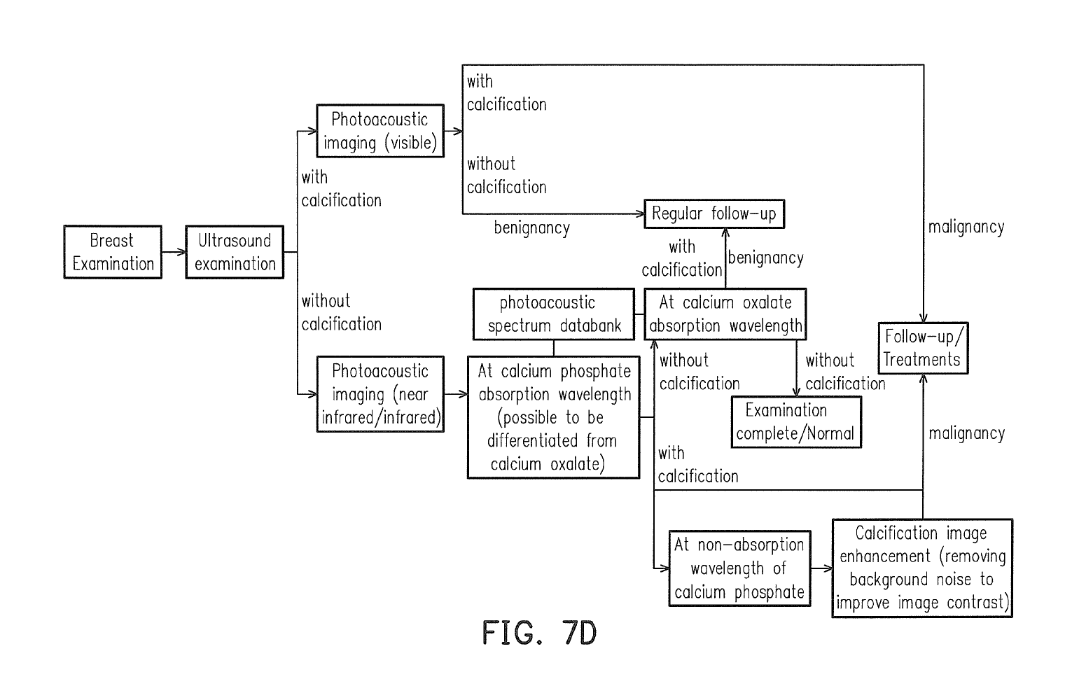

FIGS. 7A-7C show the general principles of the photoacoustic imaging method and the obtained photoacoustic images according to another embodiment of this disclosure. In FIG. 7A, the laser (shown as wavy lines) of a third wavelength is first introduced into the target 10 and the obtained photoacoustic image is shown in the right part of FIG. 7A. Later, in FIG. 7B, the laser (shown as wavy lines) of a fourth wavelength is introduced into the target 10 and the obtained photoacoustic image is shown in the right part of FIG. 7B. The large spot shown in the right part of FIG. 7A or 7B corresponds to calcification spots (such as calcium phosphate calcification or calcium oxalate calcification), while the smaller spots scattering around corresponds to noise signals (signals coming from background tissues, such as blood vessels or other soft tissues). At the third wavelength, both of the calcification spot and the noise spots are bright (exhibiting strong signals) as shown in FIG. 7A. The third wavelength may be predetermined or elected in advance from the absorption bands of the absorption spectrum of calcium phosphate or calcium oxalate, so that calcification has strong optical absorption at the third wavelength. For example, the third wavelength may be 700 nm. However, at the fourth wavelength, only the smaller noise spots are bright. The fourth wavelength may be predetermined or elected in advance from the non-absorption bands of the absorption spectrum of calcium phosphate or calcium oxalate, so that calcification has weak optical absorption at the fourth wavelength. For example, the calcium phosphate calcification has stronger optical absorption toward the third wavelength of 700 nm, instead of the fourth wavelength of 900 nm. After processing with Boolean operation, the noise signals shown in FIG. 7B is deducted from the photoacoustic image in FIG. 7A, and the resultant photoacoustic image is shown in the right part of FIG. 7C. In FIG. 7C, only the bright calcification spot is shown. By doing so, it is possible to remove the background or noise signals from the photoacoustic images and further enhance the quality of the photoacoustic image and the discriminability of the calcification spots. The third or fourth wavelength may be selected based on the type of the calcification being detected, or the surrounding tissues being measured. FIG. 7D is a flow chart illustrating the diagnosis of breast calcification using the aforementioned photoacoustic imaging method of this disclosure. According to FIG. 7D, an ultrasound examination is performed first and then the photoacoustic imaging examination(s) is performed. Similar to the diagnosis of FIG. 6B, by performing the photoacoustic image method of this disclosure, if no malignant calcification is observed at calcium phosphate absorption wavelength and no calcification is observed at calcium oxalate absorption wavelength, the examination result is normal and the examination is finished. If calcification is observed at calcium oxalate absorption wavelength, that is, benign calcification is observed, the patient may opt to follow-ups. However, if malignant calcification is observed at calcium phosphate absorption wavelength, the patient may opt to follow-ups or further treatments or the aforementioned photoacoustic imaging method may be performed at other non-absorption wavelength of calcium phosphate to enhance the calcification signal by eliminating the noise or background signals, which further confirms the observation of malignant calcification.

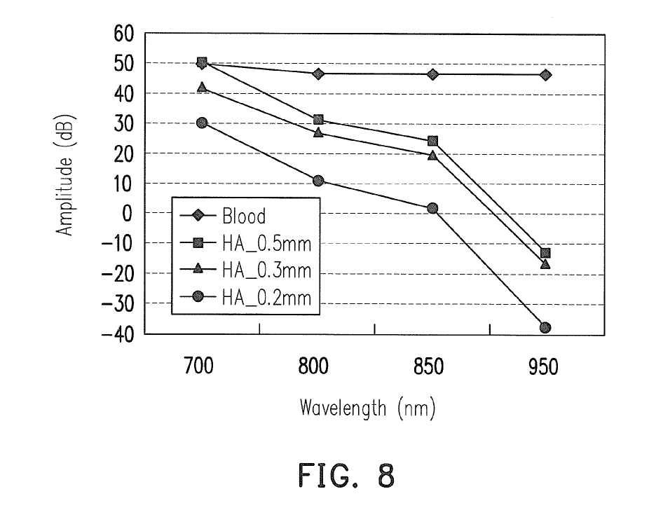

FIG. 8 is a photoacoustic spectrum of calcium phosphate samples and blood vessel. The sizes of the calcification spots for the calcium phosphate samples are 0.2 mm, 0.3 mm and 0.5 mm. The amplitudes of the photoacoustic signal are recorded as a function of the laser wavelength (the wavelength of the irradiated laser pulse). It is shown that the amplitude of the photoacoustic signal becomes smaller as the wavelength becomes longer. Further, calcification spots even small as 0.2 mm can be observed.

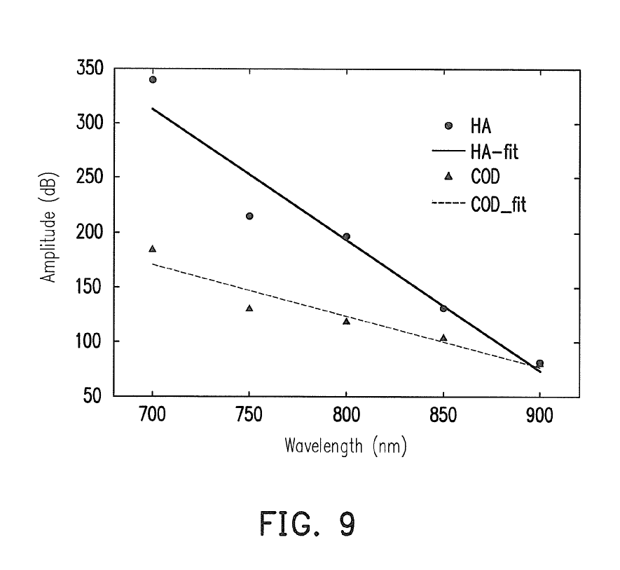

FIG. 9 is a photoacoustic spectrum of calcium phosphate and calcium oxalate. The amplitudes of the photoacoustic signal are recorded as a function of the laser wavelength (the wavelength of the irradiated laser pulse) for calcium phosphate and calcium oxalate. It is shown that the amplitude of the photoacoustic signal becomes smaller as the wavelength becomes longer and the fitted lines are shown for both samples. However, the decreasing rate (i.e. the slope of the fitted line) of the photoacoustic signal for calcium phosphate is larger than the decreasing rate of the photoacoustic signal for calcium oxalate. As shown in FIG. 9, the index (i.e. the slope of the fitted line of the photoacoustic signal) for calcium phosphate is larger than 1.5, while the index for calcium oxalate ranges from 0.5˜1.5. Though not shown in FIG. 9, the index for the noises (such as, the blood) may be smaller than 0.5. Such index, the slope of the descending fitted line, can be employed to differentiate calcium phosphate from calcium oxalate.

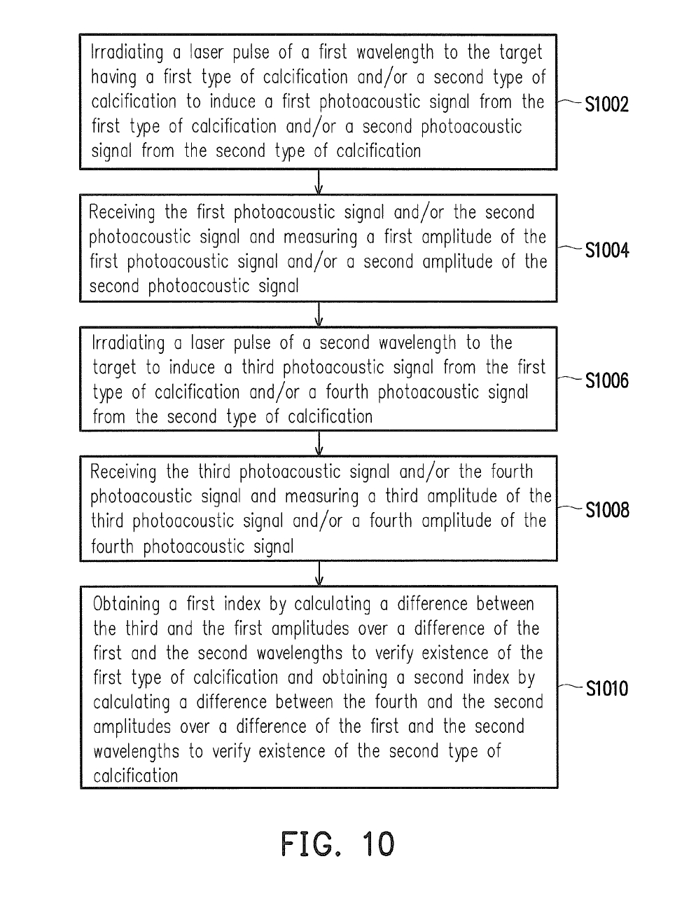

FIG. 10 is a flow chart illustrating the process steps of the photoacoustic imaging method according to another embodiment of this disclosure. In Step S1002, a laser pulse of a first wavelength is irradiated to the target having the first type of calcification and/or the second type of calcification to induce a first photoacoustic signal from the first type of calcification and/or a second photoacoustic signal from the second type of calcification. In Step S1004, the first photoacoustic signal and/or the second photoacoustic signal are received. At the same time, a first amplitude of the first photoacoustic signal and/or a second amplitude of the second photoacoustic signal are measured. In Step S1006, a laser pulse of a second wavelength is irradiated to the target having the first type of calcification and/or the second type of calcification to induce a third photoacoustic signal from the first type of calcification and/or a fourth photoacoustic signal from the second type of calcification. Generally, the first wavelength and second wavelength are in the range of visible to near-infrared light. Preferably, the first wavelength and second wavelength are in the range of 650 nm˜950 nm. For example, the first wavelength is 700 nm and the second wavelength is 900 nm. In Step S1008, the third photoacoustic signal and/or the fourth photoacoustic signal are received, and a third amplitude of the third photoacoustic signal and/or a fourth amplitude of the fourth photoacoustic signal are measured. Later, in Step S1010, a first index is obtained by calculating a difference between the third and the first amplitudes over a difference of the first and the second wavelengths to verify existence of the first type of calcification. Also, a second index is obtained by calculating a difference between the fourth and the second amplitudes over a difference of the first and the second wavelengths to verify existence of the second type of calcification.

As described herein, at least two wavelengths are selected for the calculation of the slope and more wavelengths may be employed for the calculation of the slope of the fitted line.

Taking FIG. 9 as an example, if the first and second type of calcification are respectively calcium phosphate and calcium oxalate, the first and second wavelengths are respectively 700 nm and 900 nm, the first index should be larger than 1.5, and the second index may be in the range of 0.5˜1.5.

In summary, the photoacoustic imaging method according to the embodiments of this disclosure is sensitive enough for breast cancer diagnosis and may be performed in a non-invasive way to differentiate the malignant calcification from benign calcification. In addition, the photoacoustic imaging method according to the embodiments of this disclosure is not limited to be applicable for detecting calcifications in breast tissues and may further be applied for detecting calcifications in other biological tissues or organs.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. A photoacoustic imaging method for a target, comprising:
irradiating a laser pulse of a first wavelength to the target having a first type of calcification and/or a second type of calcification to induce a first photoacoustic signal from the first type of calcification and/or a second photoacoustic signal from the second type of calcification;
receiving the first photoacoustic signal and/or the second photoacoustic signal to form a photoacoustic image, wherein the first photoacoustic signal forms a first calcification pattern in the photoacoustic image showing locations of the first type of calcification, while the second photoacoustic signal forms a second calcification pattern in the photoacoustic image showing locations of the second type of calcification; and
analyzing the photoacoustic image to verify the locations of the first type of calcification and the second type of calcification.

2. The photoacoustic imaging method of claim 1, wherein the target is a biological tissue.

3. The photoacoustic imaging method of claim 1, wherein the first type of calcification is a calcification composed of calcium phosphate (calcium phosphate calcification), and the second type of calcification is a calcification composed of calcium oxalate (calcium oxalate calcification).

4. The photoacoustic imaging method of claim 3, wherein an optical absorption of calcium phosphate calcification at the first wavelength is larger than an optical absorption of calcium oxalate calcification at the first wavelength.

5. The photoacoustic imaging method of claim 1, wherein the first wavelength is in a range of visible to near-infrared light.

6. The photoacoustic imaging method of claim 5, wherein the first wavelength is in a range of 650 nm to 750 nm.

7. A photoacoustic imaging method for a target, comprising:
irradiating a laser pulse of a first wavelength to the target having a first type of calcification and/or a second type of calcification to induce a first photoacoustic signal from the first type of calcification and/or a second photoacoustic signal from the second type of calcification;
receiving the first photoacoustic signal and/or the second photoacoustic signal to form a first photoacoustic image, wherein the first photoacoustic signal forms a first calcification pattern in the first photoacoustic image showing locations of the first type of calcification, while the second photoacoustic signal forms a second calcification pattern in the first photoacoustic image showing locations of the second type of calcification;
irradiating a laser pulse of a second wavelength to the target to induce a third photoacoustic signal from the first type of calcification and/or a fourth photoacoustic signal from the second type of calcification;
receiving the third photoacoustic signal and/or the fourth photoacoustic signal to form a second photoacoustic image, wherein the third photoacoustic signal forms a third calcification pattern in the second photoacoustic image showing locations of the first type of calcification, while the fourth photoacoustic signal forms a fourth calcification pattern in the second photoacoustic image showing locations of the second type of calcification; and
analyzing the first and second photoacoustic images to verify the locations of the first type of calcification and the second type of calcification.

8. The photoacoustic imaging method of claim 7, wherein the target is a biological tissue.

9. The photoacoustic imaging method of claim 7, wherein the first type of calcification is a calcification composed of calcium phosphate (calcium phosphate calcification), and the second type of calcification is a calcification composed of calcium oxalate (calcium oxalate calcification).

10. The photoacoustic imaging method of claim 9, wherein the first wavelength is elected from an absorption band of an absorption spectrum of calcium phosphate, so that an optical absorption of calcium phosphate calcification at the first wavelength is larger than an optical absorption of calcium oxalate calcification at the first wavelength.

11. The photoacoustic imaging method of claim 10, wherein the second wavelength is elected from an absorption band of an absorption spectrum of calcium oxalate, so that an optical absorption of calcium oxalate calcification at the second wavelength is larger than an optical absorption of calcium phosphate calcification at the second wavelength.

12. The photoacoustic imaging method of claim 7, wherein the first wavelength and second wavelength are in a range of visible to near-infrared light.

13. The photoacoustic imaging method of claim 11, wherein the first wavelength is 700 nm and the second wavelength is 900 nm.

14. The photoacoustic imaging method of claim 7, further comprising irradiating a laser pulse of a third wavelength to the target to induce a noise photoacoustic signal from a background; and
performing a Boolean operation to deduct the noise photoacoustic signal from first, second, third and/or fourth photoacoustic signals.

15. The photoacoustic imaging method of claim 14, wherein the third wavelength is elected from non-absorption bands of an absorption spectrum of calcium phosphate or calcium oxalate, so that calcium phosphate calcification or calcium oxalate calcification has weak optical absorption at the third wavelength.

16. A photoacoustic imaging method for a target, comprising:
irradiating a laser pulse of a first wavelength to the target having a first type of calcification and/or a second type of calcification to induce a first photoacoustic signal from the first type of calcification and/or a second photoacoustic signal from the second type of calcification;
receiving the first photoacoustic signal and/or the second photoacoustic signal and measuring a first amplitude of the first photoacoustic signal and/or a second amplitude of the second photoacoustic signal;
irradiating a laser pulse of a second wavelength to the target to induce a third photoacoustic signal from the first type of calcification and/or a fourth photoacoustic signal from the second type of calcification;
receiving the third photoacoustic signal and/or the fourth photoacoustic signal and measuring a third amplitude of the third photoacoustic signal and/or a fourth amplitude of the fourth photoacoustic signal; and
obtaining a first index by calculating a difference between the third and the first amplitudes over a difference of the first and the second wavelengths to verify existence of the first type of calcification and obtaining a second index by calculating a difference between the fourth and the second amplitudes over a difference of the first and the second wavelengths to verify existence of the second type of calcification.

17. The photoacoustic imaging method of claim 16, wherein the first wavelength and second wavelength are in a range of visible to near-infrared light.

18. The photoacoustic imaging method of claim 16, wherein the first wavelength and second wavelength are in a range of 650 nm˜950 nm.

19. The photoacoustic imaging method of claim 18, wherein the first wavelength is 700 nm and the second wavelength is 900 nm.
